(19) United States
(12) Patent Application Publication
(10) Pub. No.: US 2017/0367701 A1
(43) Pub. Date: Dec. 28, 2017

(54) Surgical Stapler Instrument with Auxiliary Tissue Compression Means

(71) Applicant: Chul Hi Park, Seoul (KR)

(72) Inventor: Chul Hi Park, Seoul (KR)

(21) Appl. No.: 15/632,348

(22) Filed: Jun. 25, 2017

Related U.S. Application Data
(60) Provisional application No. 62/355,272, filed on Jun. 27, 2016.

(51) Int. Cl. A61B 17/072

(52) U.S. Cl. CPC A61B 17/07207; A61B 2017/07257; A61B 2017/07278

(57) ABSTRACT

A surgical stapler instrument with an auxiliary tissue compression means for stapling a tissue with a plurality of staples comprises; a handle, an elongate shaft having a proximal end and a distal end extending therefrom, operatively coupled to said handle, and an elongate end effector having a proximal end attached to said distal end of said shaft, a distal end extending therefrom; said end effector comprising a first jaw member having an anvil thereon and a second jaw member, opposing said first jaw member, having a staple cartridge housing a plurality of staples therein and a cartridge bay configured to releasably receive said staple cartridge, and a compression bladder disposed between cartridge bay and said staple cartridge or driving at least a portion of said staple cartridge toward said first jaw member; said jaw members movable with respect to each other from an open position, wherein said jaw members are spaced apart, to a closed position wherein said jaw members are in close approximation to one another.

### CROSS REFERENCE TO RELATED APPLICATIONS

The present application claims the benefit of and priority to U.S. Provisional Patent Application No. 62/355,272, filed on Jun. 26, 2016, the entire content of which is incorporated by reference.

### TECHNICAL FIELD

The present invention relates to a medical device for use in surgical procedures. More particularly, the present invention relates to a surgical stapler instrument including a means for providing an auxiliary compression to a target tissue.

### BACKGROUND OF THE INVENTION

The utilization of mechanical tissue fastening instruments, notably, open and endoscopic surgical staplers have been increasing steadily in recent years as a substitute for suturing in joining a tissue, joining and cutting a tissue simultaneously and performing anastomosis of tubular organs belonging to the digestive system in a number of surgical disciplines. Over the years these instruments have proven to provide significant clinical benefits of improved patient outcome in addition to procedural benefits of reduced procedure time and simplified surgical tasks when compared to laborious and time consuming suturing, and related cost savings. In certain types of surgical procedures use of surgical staplers has become the preferred method of joining a tissue including the bariatric, thoracic and colorectal surgeries.

There are several known types of surgical stapler instruments specifically adapted for use in various procedures such as end-to-end anastomosis, gastrointestinal anastomosis, endoscopic gastrointestinal anastomosis, and transverse anastomosis. Examples of stapler instruments for these various procedures can be found in U.S. Pat. Nos. 5,915,616; 6,202,914; 5,865,361; and 5,964,394, which are each herein incorporated by reference. Examples of stapler instruments for use in the robot assisted surgery can be found in U.S. Pat. Nos. 8,989,903 and 8,991,678 which are each herein incorporated by reference.

Known surgical stapler instruments include an end effector that simultaneously makes a longitudinal incision in tissue and applies lines of staples on opposing sides of the incision. The end effector includes a pair of opposed jaw members, rotatably engaged with each other about a pivot at the proximal ends of the jaw members, which, if the instrument is intended for endoscopic or laparoscopic applications, are capable of passing through a cannula passageway. One of the jaw members receives a staple cartridge having at least two laterally spaced rows of staples. The other jaw member defines an anvil having staple-forming pockets aligned with the rows of staples in the cartridge. The instrument commonly includes a plurality of reciprocating wedges which, when driven distally, pass through openings in the staple cartridge and engage drivers supporting the staples to effect the firing of the staples toward the anvil.

In use, after passing through the cannula, the two jaw members, cartridge and anvil, are closed around the target tissue to be stapled and cut, and clamped to compress the tissue to a predetermined thickness in preparation for subsequent staple firing step. Proper compression of tissue is one of the factors determining viability of hemostasis and leak-free closure of the staple line formed along the cut edges of the tissue. Ideally, the tissue is to be compressed uniformly along the staple line and as close as possible to the thickness appropriate for the staple height selected by the physician in consideration of the target tissue property. In reality, the tissue compression is often non-uniform being more pronounced near the proximal end of the end effector and gradually diminishing toward the distal end thereof due to deflection and/or deformation of the anvil and, to a lesser degree, the cartridge jaw member, or a jaw member comprising a staple cartridge, induced by reactive force from the compressed tissue distributed along the length of the end effector. In instances where the target tissue is too thick for the selected staple height an insufficient or under-compression of the target tissue may result closer to the distal end of the end effector or even substantially along the full length thereof in certain cases. In addition to adversely affecting the hemostasis and sealing performances of the staple line the non-uniformity of tissue compression along the staple line may carry statistically a higher change of performance failure due to disproportionately higher load concentration, for example, during physiological motion of the organ subject to the stapling operation.

The deflection of the jaw members of end effector also increases possibility of staple malformation due to misalignment of staples in the cartridge and the staple forming pockets in the anvil, which in turn may lead to an incomplete closure of the target tissue and resultant post operative complications. In the present design of endoscopic stapler instrument the dimensions of the typically elongate jaw members comprising the end effector are constrained by the size of the cannula through which it must pass setting a limit on the stiffness thereof strongly correlated with the dimensions. Motion of the two jaw members comprising an end effector being constrained to a rotation about the pivot about which the two jaw members are engaged and the two jaw members being held stationary by the clamping torque, the reactionary translational load from the compressed tissue against the jaw members must be opposed solely by the structural stiffness of the jaw members making the dimension thereof a critical factor determining the tissue compression performance of an end effector. Due to the finite dimensions of the jaw members, deflection, particularly, that of the anvil jaw member, which is weaker of the two jaw members, is inevitable being more pronounced in a longer end effector and with a thicker tissue. In an attempt to remedy the drawbacks stemming from incomplete tissue compression and equivalently, variation in the gap distance between the two jaw members, and misalignment of the staples and staple forming pockets, in some prior art surgical stapler instrument a rigid I-beam like structural element is known to be employed during the staple firing step to forcibly and at a fairly rapid rate close up the jaw members to a predetermined gap distance and further compress the pre-compressed tissue. It is generally known that a rapid change in strain, i.e., thickness of a tissue, imposed on a viscoelastic material, which a tissue generally is, induces a high level of stress in the material, a phenomenon known as work- or strain- hardening to those of skill in the art. The induced stress typically takes time to dissipate through what is known as a relaxation process, the length of which is, among other things, specific to a given material and the physical condition subject thereto. In the case of the forcibly compressed and stapled tissue the relaxation process of this residual stress is likely to be hindered and may not occur so readily due to the staples bounding the tissue. The impact of a forcible tissue compression during a stapling operation on the clinical outcome is not fully understood but may be presumed more negative than positive.

Therefore, significant need exists for a surgical stapler instrument with a means for applying an auxiliary compression force to a tissue in ways complementary to a pre-compression by the jaw members of an end effector, and to a degree and at a rate controllable by the physician during a stapling operation.

### BRIEF SUMMARY OF THE INVENTION

The present invention relates to a medical device for use in surgical procedures. More particularly, the present invention relates to a surgical stapler instrument including a means for providing an auxiliary compression to a target tissue.

In a preferred embodiment of the present invention, a surgical stapler instrument with an auxiliary tissue compression means for stapling a tissue with a plurality of staples comprises; a handle, an elongate shaft having a proximal end and a distal end extending therefrom, said shaft operatively coupled to said handle, and an elongate end effector having a proximal end attached to said distal end of said shaft, a distal end extending therefrom; said end effector comprising a first jaw member having an anvil thereon and a second jaw member, opposing said first jaw member, having a staple cartridge housing a plurality of staples therein and a cartridge bay configured to releasably receive said staple cartridge, and a compression bladder disposed between said cartridge bay and said staple cartridge for driving at least a portion of said staple cartridge toward said first jaw member; said jaw members movable with respect to each other from an open position, wherein said jaw members are spaced apart, to a closed position wherein said jaw members are in close approximation to one another.

Methods of use in performing a surgical stapling operation with a surgical stapler instrument of the present invention are described herein. In an embodiment of the present invention a method of use includes steps of: capturing a target tissue with two jaw members comprising an end effector in an open position; clamping and compressing said target tissue by closing said two jaw members; applying an auxiliary compression to said pre-compressed target tissue by expanding a compression bladder to a predetermined extent by supplying a pressurizing fluid from an external pressurizing fluid source; collapsing said compression bladder completely by evacuating said pressurizing fluid to said external pressurizing fluid source; firing staples; and opening said two jaw members and releasing stapled and cut target tissue. In an alternate embodiment a method of use includes steps of: capturing a target tissue with two jaw members comprising an end effector in an open position; clamping and compressing said target tissue by closing said two jaw members; applying an auxiliary compression to said pre-compressed target tissue by expanding a compression bladder to a predetermined incremental extent by supplying a pressurizing fluid from an external pressurizing fluid source; collapsing said compression bladder completely by evacuating said pressurizing fluid to said external pressurizing fluid source; repeating said preceding two steps until cumulative auxiliary compression of said pre-compressed target tissue reaches a predetermined extent; firing staples; and opening said two jaw members and releasing stapled and cut target tissue. In another alternate embodiment a method of use of a surgical stapler instrument of the present invention may further include a step of removing the compression bladder prior to firing the staples by pulling on the fluid supply line connected thereto.

The presently disclosed surgical stapler instruments, together with attendant advantages, will be more clearly illustrated below by the description of the drawings and the detailed description of the embodiments.

### BRIEF DESCRIPTION OF THE DRAWINGS

The following exemplary figures are provided to supplement the description below and more clearly describe the invention. In the figures, like elements are generally designated with the same reference numeral for illustrative convenience and should not be used to limit the scope of the present invention.

FIG. 1A is a perspective view of an exemplary surgical stapler instrument according to an embodiment of the present invention.

FIG. 1B is a perspective view of an exemplary end effector of a surgical stapler instrument according to an embodiment of the present invention.

FIG. 2A is a side elevation view of a prior art end effector showing a tissue captured between an anvil and a cartridge in an open position according to an embodiment of the present invention.

FIG. 2B is a side elevation view of a prior art end effector showing a tissue in an ideally compressed state between an anvil and a cartridge in a closed position according to an embodiment of the present invention.

FIG. 2C is a side elevation view of a prior art end effector showing a tissue in a compressed state between an anvil and a cartridge in a closed position according to an embodiment of the present invention.

FIG. 3A is a perspective view of a cartridge jaw member of an end effector with a staple cartridge shown separated from a cartridge bay to show a compression bladder in a collapsed state according to an embodiment of the present invention.

FIG. 3B is a side elevation view of an end effector, similar to FIG. 2C, showing a tissue in a compressed state between an anvil and a cartridge in a closed position and a compression bladder in an expanded state according to an embodiment of the present invention.

FIG. 3C is a perspective view of a cartridge jaw of an end effector with a compression bladder in an expanded state according to an embodiment of the present invention.

FIG. 3D is a side elevation view of an end effector showing a tissue in a compressed state between an anvil and a cartridge in a closed position and a compression bladder returned to a collapsed state following an expansion, as shown in FIG. 3B, according to an embodiment of the present invention.

FIGS. 4A and 4B are perspective views of a compression bladder in an expanded and collapsed state, respectively, according to an embodiment of the present invention.

FIG. 4C is a perspective view of a compression bladder in a collapsed state according to an alternate embodiment of the present invention.

FIGS. 4D and 4E are perspective views of a compression bladder in an expanded and collapsed state, respectively, according to another alternate embodiment of the present invention.

FIG. 4F is a perspective view of a compression bladder in a collapsed state according to another alternate embodiment of the present invention.

FIG. 5A is a perspective view of a stack of film material imprinted with a seal pattern illustrating an initial step of manufacturing process of a compression bladder according to an embodiment of the present invention.

FIG. 5B is a perspective view of a seal pattern for manufacturing a compression bladder integrated with a fluid supply line and a component of an external pressurizing fluid source according to an embodiment of the present invention.

FIG. 5C is a perspective view of a composite bladder in an exploded view according to an embodiment of the present invention.

FIG. 5D is a perspective view of a non-compliant envelope according to an embodiment of the present invention.

FIG. 5E is a perspective view of a non-compliant envelope according to an alternate embodiment of the present invention.

FIG. 6A is a perspective view of an external pressurizing fluid source partially sectioned apart to show details according to an embodiment of the present invention.

FIG. 6B is a perspective view of a cartridge comprising a surgical stapler instrument according to an embodiment of the present invention.

FIG. 6C is a perspective view of a cartridge bay comprising an end effector comprising a surgical stapler instrument according to an embodiment of the present invention.

### DETAILED DESCRIPTION OF THE INVENTION

The novel features of the present invention will become apparent to those of skill in the art upon examination of the following detailed description of the invention. It should be understood, however, that the detailed description of the invention and the specific examples presented, while indicating certain embodiments of the present invention, are provided for illustration purposes only because various changes and modifications within the spirit and scope of the invention will become apparent to those of skill in the art from the detailed description of the invention and claims that follow.

Embodiments of the presently disclosed surgical instrument will now be described in detail with reference to the drawing figures wherein like reference numerals identify similar or identical elements. In the drawings and in the description which follows, the term “proximal”, as is traditional, will refer to the end of the surgical instrument which is closest to the operator while the term “distal” will refer to the end of the device which is furthest from the operator.

The present invention relates to a surgical stapler instrument for use in a surgical procedure. More particularly, the present invention relates to a surgical stapler instrument having an auxiliary compression means capable of applying compression force to the target tissue in addition to the clamping pressure applied thereto by the closure of opposing jaw members comprising an end effector of the surgical stapler instrument. The additional compression force provided by the auxiliary tissue compression means is translational in nature and substantially transverse in direction to the tissue in contrast to the clamping pressure of the jaw members powered by a rotational force about the pivot fixed in position by which the jaw members are rotatably joined and therefore highly dependent on the structural stiffness of the jaw members. The auxiliary tissue compression means comprising a surgical stapler instrument of the present invention comprises a fluid actuator powered by a pressurizing fluid, and is configured to be responsive to an external load applied thereto capable of self-regulating the distribution of output force in such a way to substantially compensate and even out the non-uniformity present in the external load within the mechanical constraint imposed by the structure of the end effector. The fluid actuator comprising the auxiliary tissue compression means is configured to be disposed in the cartridge jaw member, and directly drive the staple cartridge toward the captured and compressed tissue, and the anvil jaw member providing additional compression to the tissue. The auxiliary tissue compression means of the present invention helps substantially reduce, if not eliminate, the incompleteness and non-uniformity of tissue compression and variation in the gap distance between anvil and cartridge jaw members along the length of an end effector often found in a prior art surgical stapler instrument. Although the implementation and operation of an auxiliary tissue compression means in various embodiments of the present invention will be described in the following as it relates to a surgical stapler instrument, it should be apparent to those of skill in the art that the aspects of the present disclosure may be readily adapted for use with other types of surgical instruments.

The present invention is being discussed in terms of endoscopic procedures and apparatus. However, use herein of terms such as “endoscopic”, should not be construed to limit the present invention to a surgical stapler instrument for use only in conjunction with an endoscopic tube (i.e., trocar). On the contrary, it is to be understood that the present invention may find use in any procedure where access is limited to a small incision, including but not limited to laparoscopic procedures, as well as open procedures.

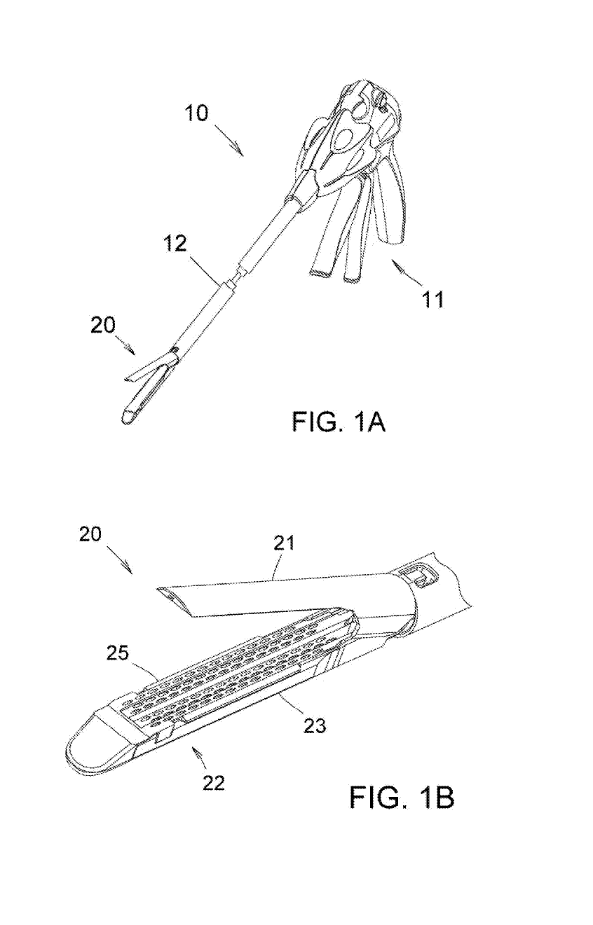

Referring to FIGS. 1A and 1B, an exemplary prior art surgical stapler instrument 10 and an enlarged view of an end effector 20 thereof are shown. Surgical stapler instrument 10 includes a handle 11, an elongate end effector 20 and an elongate tube 12 that operatively connects handle 11 and end effector 20. End effector 20 comprises a first jaw member 21 and a second jaw member 22 extending from proximal to distal end defining a length thereof. Second jaw member 22 comprises an elongate channel or a cartridge bay 23 configured to releasably receive a staple cartridge 25 housing a plurality of staples and first jaw member 21 comprises an anvil that is aligned with and pivotally attached to cartridge bay 23 forming a pair of opposed jaw members that open and close, when operated on with handle 11 by the physician, to capture and compress a target tissue there-between for stapling. End effector 20 is configured in such a way that jaw members 21, 22 are capable of rigidly holding their approximated positional relationship when closed to compress the target tissue prior to and during the firing of staples.

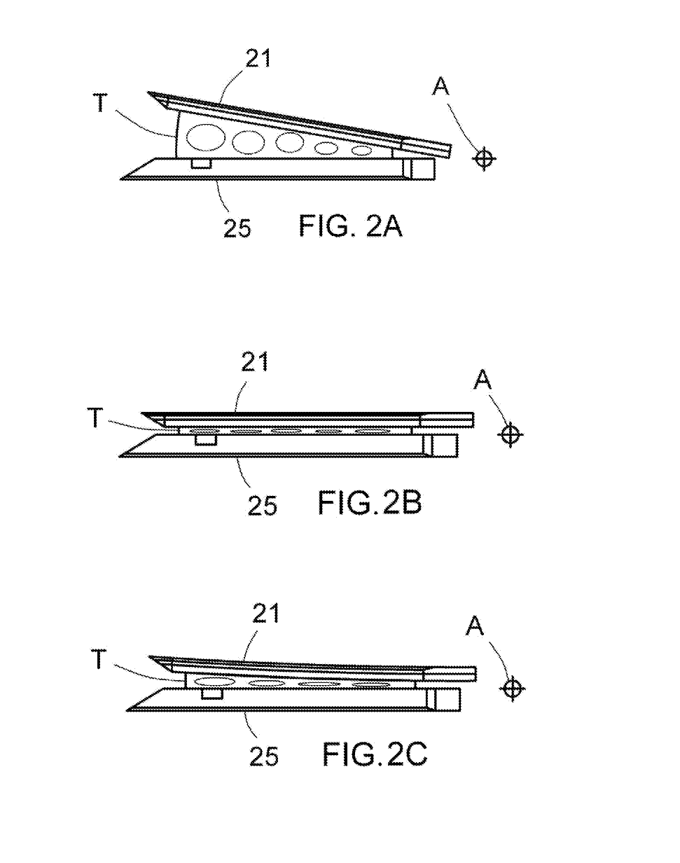

Referring to FIGS. 2A-2C, schematically illustrating, in a side elevation view, the operations of an end effector of a prior art surgical stapler instrument, represented by an anvil 21 and a staple cartridge 25, in capturing and compressing a target tissue T, in an initial step target tissue T to be stapled and cut is captured and positioned between anvil 21 and staple cartridge 25 in an open position about a pivot axis shown as a crosshair centered on a circle, indicated by letter A. As shown in the Figures the direction of pivot axis A is perpendicular to the view. It is to be noted that pivot axis A is located proximal to the proximal end of staple cartridge 25. In certain stapler instrument designs the pivot axis of an end effector is configured to translate along a predetermined path in opening and closing jaw members comprising the end effector but still remains proximal to the proximal end of a staple cartridge throughout the translation. After positioning target tissue T the physician operates the handle to close anvil 21 and staple cartridge 25 in order to compress target tissue T, and bring the thickness of target tissue T and the gap distance between anvil 21 and staple cartridge 25 to a predetermined value approximating the formed height of the staple selected by the physician. Ideally the thickness of compressed target tissue and the gap distance should be uniform along the full length of the end effector as illustrated in FIG. 2B. In reality a more likely result of the compression operation is that anvil 21 and staple cartridge 25 are deflected or deformed under reactionary force from compressed target tissue T distributed over the length of the portion of the end effector in contact with target tissue T, more likely so for anvil 21 than staple cartridge 25 mainly due to difference in structural rigidity. As illustrated in FIG. 2C, the resultant non-uniformities in the compressed tissue thickness and the gap distance are such that they become larger and consequently, tissue compression becomes weaker going from proximal to distal ends of the end effector. This effect is generally more pronounced in a longer end effector and with a thicker target tissue.

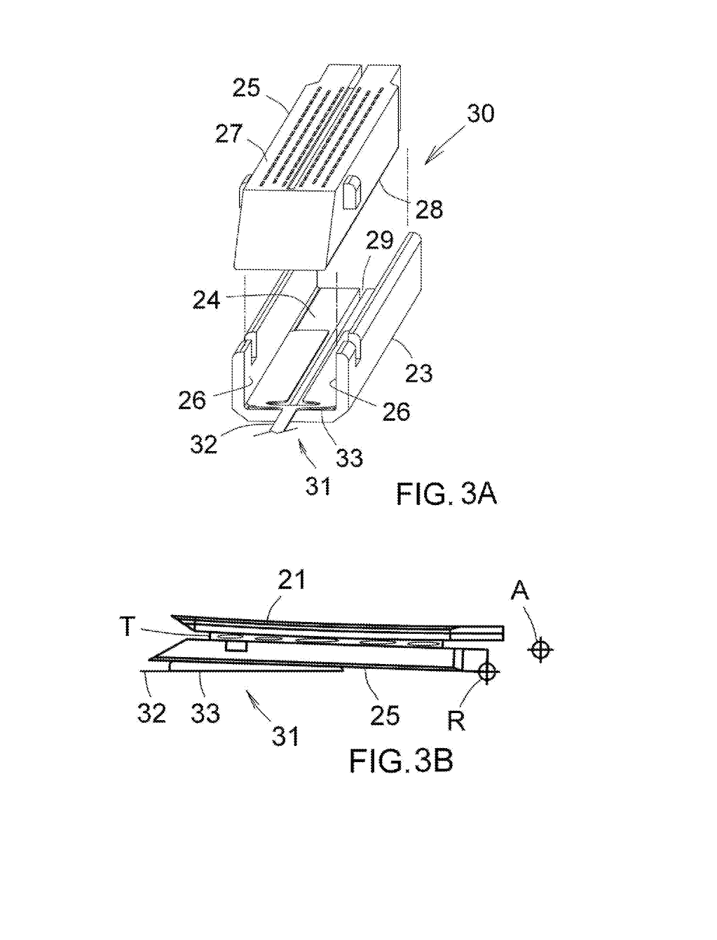

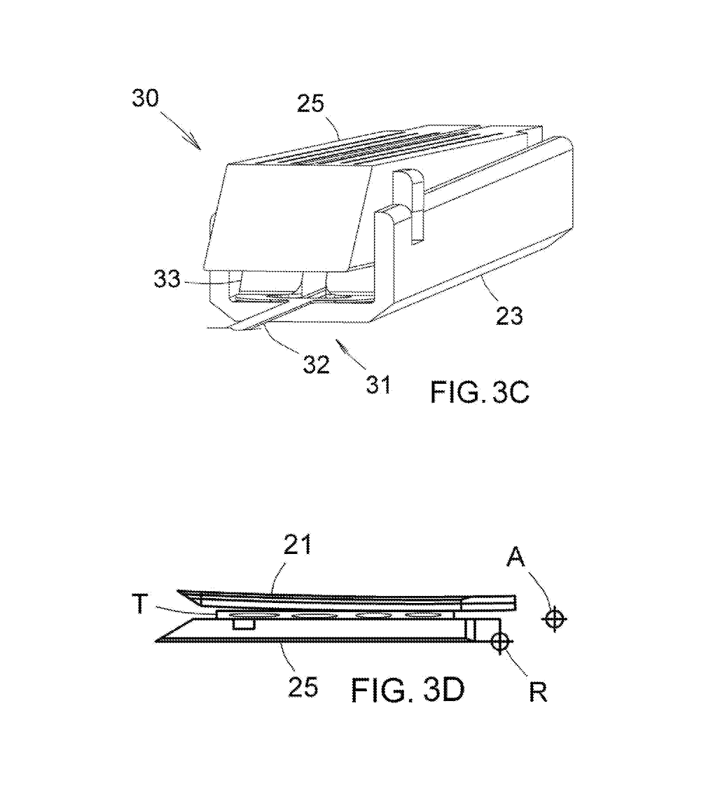

Referring to FIG. 3A schematically showing, in an exploded view, one of the jaw members of an end effector, of a surgical stapler instrument of the present invention, in a preferred embodiment, a jaw member 30 comprises a cartridge bay 23 having a floor 24, a channel 29 and two walls 26, a staple cartridge 25 having a top or tissue contacting surface 27 and a bottom surface 28, housing a plurality of staples (not shown in the Figure), and a compression bladder 31 having a fluid passage 32 and a body 33, disposed between bottom surface 28 of staple cartridge 25 and floor 24 of cartridge bay 23. In an embodiment compression bladder 31 may be configured to be expanded and collapsed by supply and evacuation of a pressurizing fluid through a fluid supply line (not shown in the Figure) to and from an external pressurizing fluid source (not shown in the Figure). Referring to FIGS. 3B and 3C showing, in a side elevation view, an end effector of a surgical stapler instrument of the present invention comprising an anvil 21, a staple cartridge 25 and a compression bladder 31 (shown in an expanded state), and, in a perspective view, a jaw member 30 of a surgical stapler instrument of the present invention comprising a cartridge bay 23, respectively, a staple cartridge 25 and a compression bladder 31 (shown in an expanded state), when expanded compression bladder 31 exerts a force on a portion of bottom surface 28 of staple cartridge 25 in contact therewith. This force of expansion is powered by the internal fluid pressure of compression bladder 31 and the direction thereof is substantially perpendicular to the length of staple cartridge 25 causing translation thereof toward tissue T pre-compressed by closure of two jaw members comprising an end effector. As will be described later in this Description the configuration and construction of compression bladder 31 is such that, expansion of compression bladder 31 occurs in such a way to approximately complement and substantially even out the non-uniform distribution of the reactionary force from non-uniformly pre-compressed target tissue T, as described previously, to staple cartridge 25, as schematically illustrated in FIG. 3B, the expansion of compression bladder 31 being more pronounced distally than proximally. Since staple cartridge 25 is substantially rigid along the length thereof, the force of expansion of compression bladder 31 acts substantially to rotate staple cartridge 25 with respect to a pivot axis located near the heel of staple cartridge 25, shown as a crosshair centered on a circle and indicated by letter R in FIG. 3B, the direction of which is perpendicular to the view. Since this pivot axis R is distal to pivot axis A of two jaw members comprising the end effector the rotation of staple cartridge 25 results in reduction of the gap distance between anvil 21 and staple cartridge 25 and further compression of pre-compressed target tissue T, more so distally than proximally. As schematically illustrated in FIG. 3D, immediately following expansion and collapse of compression bladder 31 the thickness of target tissue T become more uniform than prior to an auxiliary compression by compression bladder 31 although anvil 21 may still bear a curvature of a varying degree, albeit smaller than prior to an auxiliary compression, because the reactionary force from compressed tissue T is still present along the portion of end effector near the proximal end thereof.

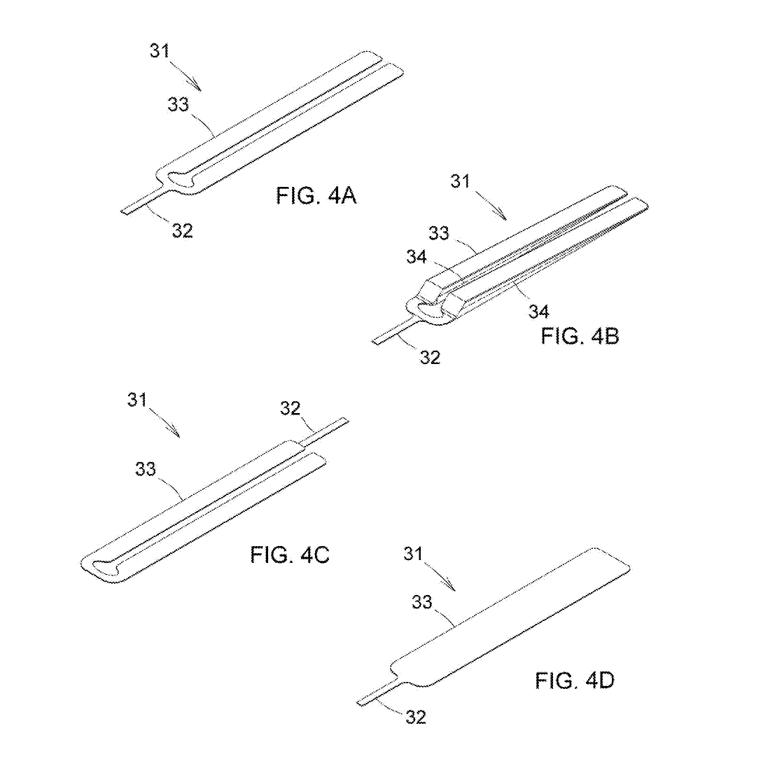

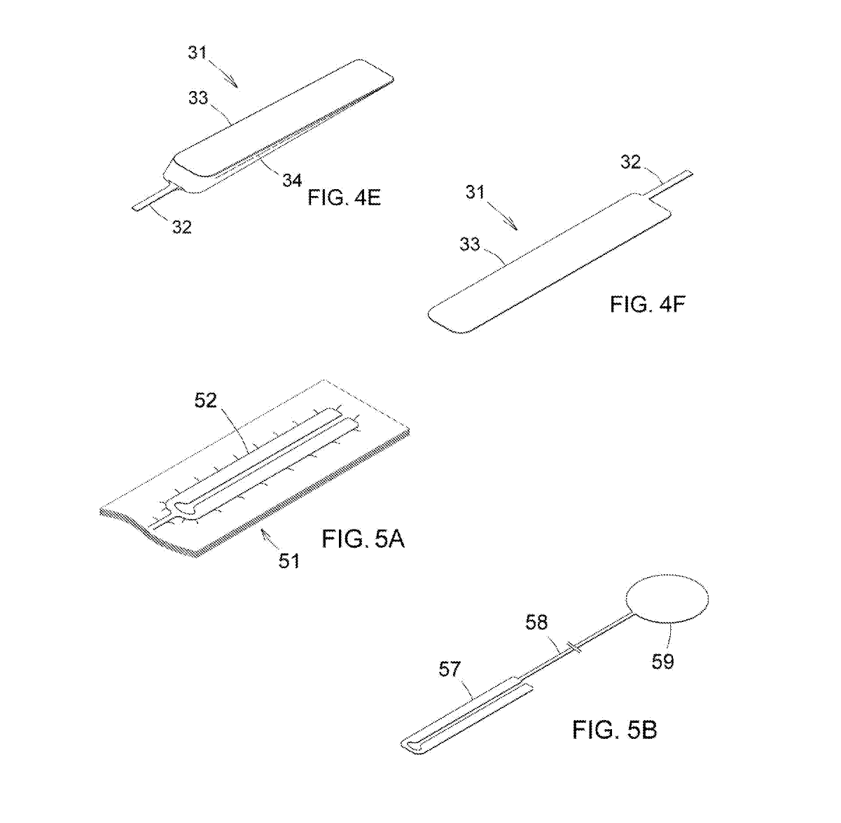

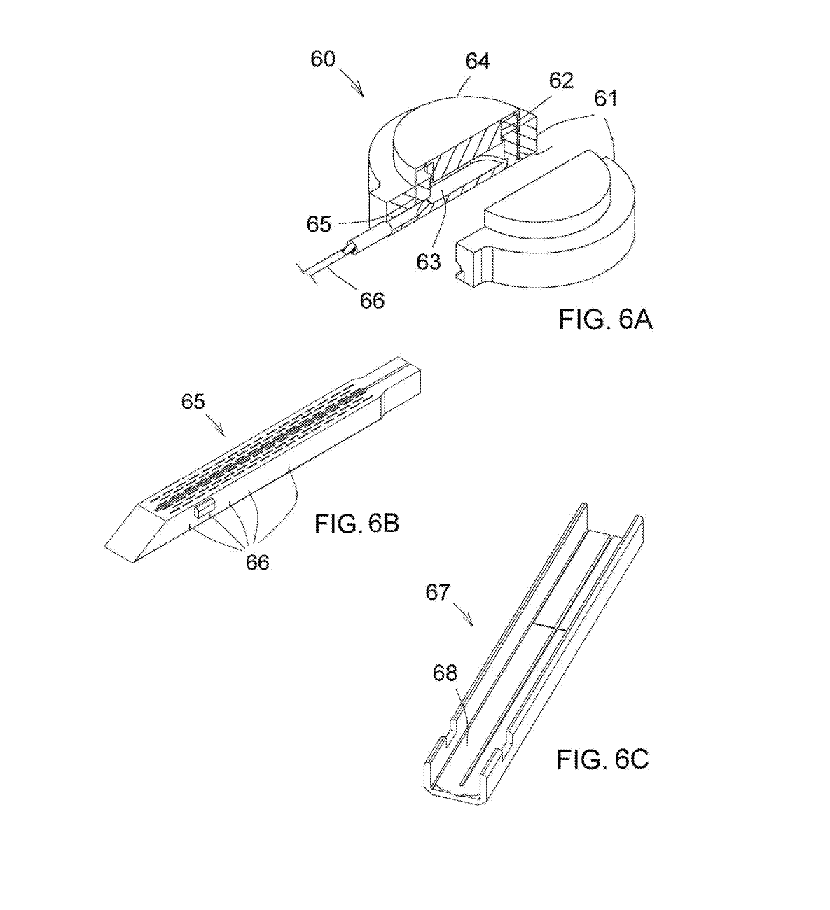

Referring to FIGS. 4A and 4D schematically showing, in a perspective view, compression bladders in a collapsed state configured for use in a cartridge bay with and without a channel (for example, as shown in FIG. 3A indicated by numeral 29), respectively, and to FIGS. 4B and 4E showing the compression bladders in an expanded state, in embodiments of the present invention, compression bladder 31 in a collapsed state may be configured to have a shape and dimensions, in a plane parallel to floor 24, for at least body 33 thereof to be fully enclosed between bottom surface of 28 of staple cartridge 25 and floor 24 of cartridge bay 23. Preferably, body 33 of compression bladder 31 may be dimensioned, in width, to span substantially the full width of floor 24 between two walls 26 covering the closed portion thereof and, in length, to extend a predetermined portion of floor 24 along the length of cartridge bay 23. In an alternate embodiment the width of body 33 of compression bladder 31 may be dimensioned to cover only a predetermined portion of the full width of floor 24. In an embodiment the thickness of compression bladder 13 in a collapsed state may be configured to be as thin as practically possible in order to minimize impact on the overall dimension of the end effector in direction substantially perpendicular to the length thereof. As will be described with regard to FIG. 6C, a space may be provided in the end effector of a surgical stapler instrument of the present invention for compression bladder 31 in a collapsed state to reside without altering existing profile of, for example, prior art surgical stapler instruments where compression bladder 31 is retrofitted to provide an auxiliary tissue compression capability in an embodiment of the present invention.

In an embodiment of the present invention, being fully enclosed between bottom surface of 28 of staple cartridge 25 and floor 24 of cartridge bay 23 body 33 of compression bladder 31 is supported, during expansion thereof, on four sides by two walls 26 and floor 24 of cartridge bay 23 and bottom surface 28 of staple cartridge 25. The proximal end of body 33 is also closed in by bottom surface 28 and floor 24 being constantly in contact during the motion of staple cartridge 25 driven by expansion of compression bladder 31 as described previously. On the other hand the distal end of body 33 is open and expansion thereof in that direction is unsupported. The internal fluid pressure acting on this unsupported portion does not contribute to driving the motion of staple cartridge 25 and, to some degree, would hinder expansion of compression bladder 31 in direction toward staple cartridge 25, if left unchecked, by counteracting, through expansion in the distal direction, the driving force substantially equal to the internal fluid pressure force acting the portion of area of bottom surface 28 in contact with compression bladder 31. Therefore it is advantageous to maximize this contact area and contain the distal expansion of the unsupported portion of compression bladder 31, which would allow operation of compression bladder 31 at that much lower internal fluid pressure.

In an embodiment of the present invention fluid passage 32 of compression bladder 31 may be located at the distal end thereof, as shown in FIGS. 4A and 4D, to be connected to an external pressurizing fluid source (not shown in the Figures) disposed extracorporeally through a fluid supply line (not shown in the Figures) passing through a cannula. In an alternate embodiment, fluid passage 32 of compression bladder 31 may be located at the proximal end thereof, as shown in FIGS. 4C and 4F, to be connected to a fluid supply line (not shown in the Figures) routed through elongate tube 12 to handle 11 (for example, such as shown in FIG. 1A) and connected to an external pressurizing fluid source (not shown in the Figures). In another alternate embodiment, fluid passage 32 may be passed through channel 29 in cartridge bay 23 (shown in FIG. 3A) and connected through a fluid supply line to an external pressurizing fluid source (not shown in the Figures). In an embodiment a fluid supply line may comprise a tube with a solid wall or a lay-flat tube. In an alternate embodiment a fluid supply line may be constructed integrated with a compression bladder as will be described hereinafter. In an embodiment of the present invention a pressurizing fluid for expanding a compression bladder may be a liquid including a saline solution, glycerin, silicone oil, water, de-ionized and distilled water or a mixture thereof. In an alternate embodiment a pressurizing fluid may be a gas including air and carbon dioxide. In an embodiment the operating internal pressure of the compression bladder may be between 0.2 atm and 10 atm. Preferably, the operating internal pressure may be between 0.5 atm and 2 atm. In an embodiment an applicator of a predetermined configuration may be employed to consistently position a compression bladder in a cartridge bay of end effector of a surgical stapler instrument of the present invention.

In a preferred embodiment of the present invention the compression bladder comprising a surgical stapler instrument may be made of a semi-compliant or non-compliant polymeric film material selected from a group including LDPE (low density polyethylene), HDPE (high density polyethylene), Nylon, PVC (polyvinyl chloride), PET (polyethylene terephthalate), PEVAC (polyethylene-co-vinyl acetate-co-carbon monoxide), PEBAX (polyether block amide) and Polyurethane and a predetermined blend thereof and is configured to be able to resist a blow-out of the body of the compression bladder on the unsupported portion thereof or through channel 29 (shown in FIG. 3A) that may be present in the cartridge bay and/or a corresponding channel in the staple cartridge. A semi-compliant or non-compliant polymeric material is generally resistant to stretching under tensile stress loads, for example, the ones on the compression bladder developed by the internal pressure of the compression bladder. Also, a bladder made of such materials are capable of resisting significant changes from the design shape and dimensions under internal pressure therein exceeding a predetermined optimum operating internal pressure to a limit. Preferably, the compression bladder may be made of a polymeric film material of high tensile strength. A bladder made of such materials generally exhibits a high burst rating, that is, the highest operating internal pressure to which the bladder may be safely expanded without a significant risk of rupture, and may be operated at internal pressure at level higher than normally acceptable otherwise, which generally helps improve the mechanical performances of the compression bladder by providing enhanced structural rigidity thereof. Preferably, the compression bladder made of a polymeric film material may be manufactured to a final design configuration using a blow molding method well known to those of skill in the art. Alternately, the bladder may be manufactured using methods including vacuum forming, compression molding and dip molding. Preferably, a plurality of fold lines of predetermined configuration (indicated by numeral 34 in FIGS. 4B and 4E in an exemplary embodiment) may be introduced to the compression bladder during manufacturing process to promote orderly pleating thereof transitioning from expanded to collapsed state. In an embodiment the thickness of a film material comprising the compression bladder may be between 0.01 mm and 0.5 mm. Preferably, the thickness may be between 0.015 mm and 0.25 mm. In an embodiment the compression bladder may be constructed out of a plurality of polymeric films of one or more compliance characteristics including compliant, semi-compliant and non-compliant, which would enable selectively modifying the compliance and/or tensile characteristics of the compression bladder without sacrificing the flexibility thereof.

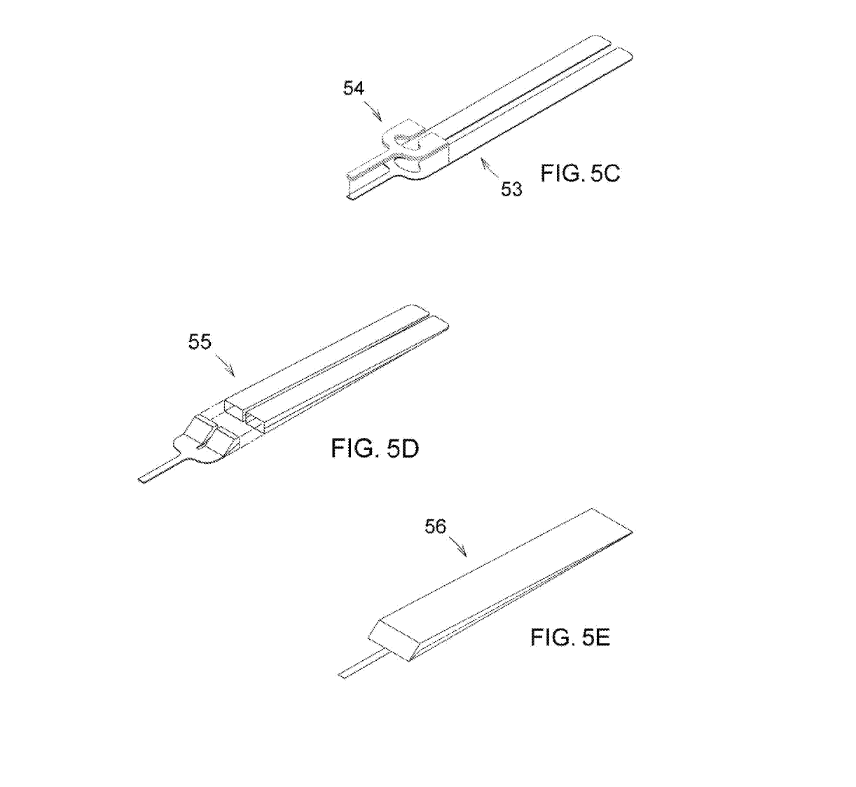

In a preferred embodiment of the present invention a compression bladder comprising a surgical stapler instrument may be made up of a plurality of semi-compliant or non-compliant polymeric film material to improve flexibility and folding characteristics of the compression bladder while retaining superior compliance characteristic provided by a film of the same kind and of thickness comparable to the combined thickness of the plurality of film material. Preferably, the compression bladder is manufactured first by imprinting a fluid-tight seal of a predetermined pattern on a stack of polymeric film materials using methods well known to those of skill in the art including thermal welding using a heated stamp mold, adhesive bonding, noncontact heat sealing methods using ultrasound and laser as an energy source, and other methods of mechanical joining of polymeric film material. In an embodiment FIG. 5A illustrates this manufacturing step showing a stack of polymeric films 51 and a seal pattern 52 imprinted thereon. In an embodiment the final design configuration of the compression bladder is then produced in a mold, whose cavity shape and dimension approximates the largest extent the compression bladder is expected to expand, by blow molding the imprinted polymeric film material. In an alternate embodiment any other methods well known to those of skill in the art including vacuum forming, dip molding and compression molding to produce the final design configuration of the compression bladder. One of notable advantages provided by the described manufacturing method is that a fluid supply line may be manufactured integrated with a compression bladder by including a seal pattern defining a fluid supply line on appropriately dimensioned stack of polymeric film material in laying out seal pattern for a compression bladder in an embodiment of the present invention, as schematically illustrated in FIG. 5B showing a perspective view of a seal pattern for manufacturing a composite bladder 57 integrated with a fluid supply line 58 and a component 59 of an external pressurizing fluid source. A fluid supply line integrated with a compression bladder obviates need for a rigid and bulky tube fitting, and is of a low profile lay-flat type that can readily pass through a cannula. In an alternate embodiment certain fluid handling components of an external pressurizing fluid supply may be manufactured integrated with a compression bladder and a fluid supply line using the same manufacturing method potentially enabling construction of a unitary fluid system as schematically illustrated in FIG. 5B. In an embodiment the compression bladder may comprise a plurality of compartments in fluid communications with one another to enhance structural integrity and mechanical performance thereof during expansion.

In an alternate embodiment of the present invention a compression bladder may be configured as a composite bladder comprising a plurality of zones with varying compliance characteristics. For example a compression bladder may be configured to be substantially non-compliant on the unsupported portion and the portions neighboring the channels in the cartridge bay and staple cartridge while leaving the rest to be substantially compliant providing a potential advantage of generally reduced overall thickness and enhanced folding characteristics of a compression bladder. As an example, a compression bladder with two compliance zone, substantially compliant and non-compliant, may be manufactured by adding addition step to the manufacturing method described in the previous section of applying additional polymeric film material to the zone where higher stiffness or substantial non-compliance is needed such as the unsupported portion of a compression bladder as schematically illustrated in FIG. 5C showing added films 54 separated from a primary stack of films 53 for fabricating a compression bladder.

In another alternate embodiment of the present invention, the compression bladder may be of composite structure comprising an expandable/collapsible bladder component made of compliant elastomeric material and a substantially semi-compliant or non-compliant envelope defining maximum extent the compliant bladder may expand. The elastomeric material for construction of the bladder component may be selected from a group including natural, silicon, neoprene and other rubber material well known to those of skill in the art. In an embodiment the bladder component may be configured to be of smaller profile than the final design configuration of the compression bladder. In an alternate embodiment the bladder component may be configured to take on the shape of substantially fully expanded compression bladder. In an embodiment the envelope of the composite structure bladder may be made of a very thin, for example, 0.0005″ thick PET film, to a shape approximating the largest extent of expansion of the compression bladder as shown in FIGS. 5D (shown disassembled) and 5E indicated by numeral 55 and 56, respectively.

In a preferred embodiment of the present invention an external pressurizing fluid source is a manually operated fluid cylinder as schematically illustrated in FIG. 6A shown sectioned apart to show details. In an embodiment an external pressurizing fluid source 60, connected with a fluid supply line 66, may comprise a cylinder body 61 housing a cylindrical cavity 63, a piston 62 configured to reciprocate in cavity 63 and a piston guide 64 joined with piston 62 and configured to reciprocate along a guide slot 65 disposed in cylinder body 61 defining a straight path for piston motion and a stable platform to which a force powering external pressurizing fluid source 60 is applied. Preferably, a physician operating a surgical stapler instrument of the present invention may power external pressurizing fluid source 60 by pressing down with the thumb on piston guide 64. The reactionary force from a tissue being compressed is directly fed back to piston 62 and piston guide 64, and to the thumb of physician enabling tactile assessment of condition of the tissue under compression. In an alternate embodiment the fluid cylinder may be replaced by a bladder to simplify the construction and enhance sensitivity to the force feedback from the tissue by eliminating friction that may be present between a piston seal and the wall of the cylindrical cavity. In another alternate embodiment an external pressurizing fluid source may be of electro-mechanical unit controlled by a computer. In an embodiment an external pressurizing fluid source or a main control function thereof may be integrated with the handle of a surgical stapler instrument of the present invention for ease of operation. Referring to FIG. 6B, in an embodiment of the present invention a staple cartridge 65 may be modified to include, for example, a plurality of slits 66 substantially perpendicular to the length disposed on the bottom surface thereof to enable controlled bending thereof during expansion of a compression bladder. In an alternate embodiment a staple cartridge or a portion thereof may be made of material having a predetermined flexibility to be able to flex under actuation by expansion of a compression bladder. As illustrated in FIG. 6C a cartridge bay 67 may include a recess 68 for stowage of a compression bladder in a collapsed state in an embodiment of the present invention.

In embodiments of the present invention the extent of auxiliary compression with a compression bladder comprising a surgical stapler instrument may be controlled in a number of ways. In an embodiment the shape and dimension of a substantially non-compliant compression bladder may be predetermined to impose a substantially hard limit on the expansion thereof and correspondingly the extent of motion of the staple cartridge. In an alternate embodiment the expansion of a compression bladder may be limited by regulating the amount of pressurizing fluid supplied from an external pressurizing fluid source. In another alternate embodiment, the end effector may be configured to provide a physical stop to the motion of the staple cartridge under actuation by the compression bladder. In still another alternate embodiment of the present invention, the end effector may be further configured to include features for guiding the motion of the staple cartridge to promote an orderly reseating thereof.

Methods of use in performing a surgical stapling operation with a surgical stapler instrument of the present invention are described herein. In an embodiment of the present invention a method of use includes steps of: capturing a target tissue with the two jaw members of end effector in an open position, as illustrated in FIG. 2A; clamping and compressing the target tissue by closing the two jaw members, as illustrated in FIG. 2C; applying an auxiliary compression to the pre-compressed target tissue by expanding the compression bladder to a predetermined extent by supplying a pressurizing fluid from an external pressurizing fluid source, as illustrated in FIG. 3B; collapsing the compression bladder completely by evacuating the pressurizing fluid to the external pressurizing fluid source, as illustrated in FIG. 3D; firing the staples; and opening the two jaw members and releasing the stapled and cut target tissue. In an alternate embodiment a method of use includes steps of: capturing a target tissue with the two jaw members of end effector in an open position; clamping and compressing the target tissue by closing the two jaw members; applying an auxiliary compression to the compressed target tissue by expanding the compression bladder to a predetermined incremental extent by supplying a pressurizing fluid from an external pressurizing fluid source; collapsing the compression bladder completely by evacuating the pressurizing fluid to the external pressurizing fluid source; repeating the previous two steps until the cumulative auxiliary compression of the pre-compressed target tissue reaches a predetermined extent; firing the staples; and opening the two jaw members and releasing the stapled and cut target tissue. In another alternate embodiment a method of use of a surgical stapler instrument of the present invention may further include a step of removing the compression bladder prior to firing the staples by pulling on the fluid supply line connected thereto.

Although the implementation and operation of an auxiliary tissue compression means in various embodiments of the present invention is described above as it relates to a cartridge jaw member comprising a staple cartridge of an end effector of a surgical stapler instrument of the present invention, it should be apparent to those of skill in the art that the aspects of the present disclosure may be readily adapted to an anvil jaw member comprising an anvil. For example, an anvil jaw member may be configured to comprise two structural members fixedly joined or rotatably joined with respect to a pivot at the proximal end thereof enabling relative motion thereof which may be a flexure of one or both members with respect to each other and a rotation of one of the members with respect to the other, respectively. A compression bladder may be positioned between the two members to effect an auxiliary compression in a manner described above in relation to the cartridge jaw member of the end effector.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

While preferred illustrative embodiments of the invention are described above, it will be apparent to those skilled in the art that various changes and modifications may be made therein without departing from the invention. Accordingly, the appended claims should be used to interpret the scope of the present invention.

What is claimed is:

1. A surgical stapler instrument with an auxiliary tissue compression means for stapling a tissue with a plurality of staples comprising:
a handle;
an elongate shaft having a proximal end and a distal end extending from said handle, and operatively coupled to said handle;
an elongate end effector having a proximal end attached to said distal end of said shaft, a distal end extending therefrom;
said end effector comprising a first jaw member having an anvil thereon and a second jaw member, opposing said first jaw member, comprising a staple cartridge having a tissue contacting surface and a bottom surface housing a plurality of staples therein and a cartridge bay having a floor and two walls configured to releasably receive said staple cartridge, and a compression bladder having a fluid passage and a body disposed between said floor of said cartridge bay and said bottom surface of said staple cartridge for driving at least a portion of said staple cartridge toward said first jaw member;
wherein said jaw members movable with respect to each other from an open position wherein said jaw members are spaced apart, to a closed position wherein said jaw members are in close approximation to one another.

2. A surgical stapler instrument of claim 1, wherein said body of said compression bladder is dimensioned, in width, to span substantially the full width of said floor between said two walls comprising said cartridge bay covering the closed portion thereof and, in length, to extend a predetermined portion of said floor along the length of said cartridge bay.

3. A surgical stapler instrument of claim 1, wherein said body of said compression bladder is dimensioned, in width, to cover a predetermined portion of the full width of said floor comprising said cartridge bay.

4. A surgical stapler instrument of claim 1, wherein said compression bladder is made of a semi-compliant or non-compliant polymeric film material selected from a group including LDPE (low density polyethylene), HDPE (high density polyethylene), Nylon, PVC polyvinyl chloride), PET (polyethylene terephthalate), PEVAC (polyethylene-co-vinyl acetate-co-carbon monoxide), PEBAX (polyether block amide) and Polyurethane and a predetermined blend thereof.

5. A surgical stapler instrument of claim 1, wherein said compression bladder comprises a plurality of compartments in fluid communications with one another.

6. A surgical stapler instrument of claim 1, wherein said compression bladder is made up of a plurality of semi-compliant polymeric film material.

7. A surgical stapler instrument of claim 1, wherein said compression bladder is made up of a plurality of non-compliant polymeric film material.

8. A surgical stapler instrument of claim 1, wherein said compression bladder is of composite structure comprising an expandable and collapsible bladder component made of compliant elastomeric material and a substantially semi-compliant envelope defining maximum extent said compliant bladder may expand.

9. A surgical stapler instrument of claim 1, wherein said compression bladder is of composite structure comprising an expandable and collapsible bladder component made of compliant elastomeric material and a substantially non-compliant envelope defining maximum extent said compliant bladder may expand, respectively.

10. Method of use of a surgical stapler instrument with an auxiliary tissue compression means of the present invention includes steps of:
capturing a target tissue with two jaw members comprising an end effector in an open position;
clamping and compressing said target tissue by closing said two jaw members;
applying an auxiliary compression to said compressed target tissue by expanding a compression bladder to a predetermined extent by supplying a pressurizing fluid from an external pressurizing fluid source;
collapsing said compression bladder completely by evacuating said pressurizing fluid to said external pressurizing fluid source;
firing staples; and
opening said two jaw members and releasing stapled and cut target tissue.

11. Method of use of a surgical stapler instrument with an auxiliary tissue compression means of the present invention includes steps of:
capturing a target tissue with two jaw members comprising an end effector in an open position;
clamping and compressing said target tissue by closing said two jaw members;
applying an auxiliary compression to said compressed target tissue by expanding a compression bladder to a predetermined incremental extent by supplying a pressurizing fluid from an external pressurizing fluid source;
collapsing said compression bladder completely by evacuating said pressurizing fluid to said external pressurizing fluid source;
repeating said two preceding steps until cumulative auxiliary compression of said compressed target tissue reaches a predetermined extent;
firing staples; and
opening said two jaw members and releasing stapled and cut target tissue.
